(19) United States
(12) Patent Application Publication
(10) Pub. No.: US 2006/0147932 A1
(43) Pub. Date: Jul. 6, 2006

(54) Methods of screening for modulators of nerve growth factor

(76) Inventors: Alun Davies, St. Andrews (GB); Jan Markus Grimm, Menlo Park, CA (US); Sean Wyatt, Mountain View, CA (US)

Correspondence Address: MORRISON & FOERSTER LLP, 755 PAGE MILL RD, PALO ALTO, CA, 94304-1018, US

(21) Appl. No.: 10/542,184

(22) PCT Filed: Jan. 20, 2004
(86) PCT No.: PCT/US04/01609

§ 371 Date: Feb. 6, 2006

Related U.S. Application Data
(60) Provisional application No. 60/441,070, filed on Jan. 18, 2003.

(51) Int. Cl. C12Q 1/68; G01N 33/567

(52) U.S. Cl. 435/6; 435/7.2

(57) ABSTRACT

The invention relates, in general, to a method of screening for agents that modulate NGF activity. More specifically this invention provides a method of assessing the ability of a candidate agent to modulate NGF activity comprising, measuring the level of expression of one or more or two or more NGF responsive genes in a culture of neurons expressing the high-affinity trk A receptor after contact with a candidate agent. The invention further provides methods of culturing primary cultures of neurons expressing the high-affinity trk A receptor and methods of isolating polynucleotides from such cultures.

### RELATED APPPLICATIONS

This application claims priority under 35 U.S.C. §119(e) to U.S. Patent Application 60/441,070 filed Jan. 18, 2003, the contents of which is herein incorporated by reference in its entirety.

### STATEMENT REGARDING FEDERALLY SPONSORED RESEARCH OR DEVELOPMENT

Not applicable.

### FIELD OF THE INVENTION

This application is in the field of neurotrophic growth factors, in particular this invention relates to methods for screening for modulators of Nerve Growth Factor (NGF) activity.

### BACKGROUND OF THE INVENTION

Nerve growth factor (NGF) was the first neurotrophin identified, and its role in the development and survival of both peripheral and central neurons has been well characterized. NGF has been shown to be a critical survival and maintenance factor in the development of peripheral sympathetic and embryonic sensory neurons and of basal forebrain cholinergic neurons (Smeyne, et al., Nature 368:246-249 (1994); Crowley, et al., Cell 76: 1001-1011 (1994)). NGF upregulates expression of neuropeptides in sensory neurons (Lindsay, et al., Nature 337:362-364 (1989)) and its activity is mediated through two different membrane-bound receptors. The TrkA tyrosine kinase receptor mediates high affinity binding and the p75 receptor, which is structurally related to other members of the tumor necrosis factor receptor family, mediates low affinity binding (Chao, et al., Science 232:518-521 (1986)).

One of NGF's roles in the nervous system is in modulation of pain and pain sensitivity. For example, injection of NGF leads to a significant increase in pain and pain sensitivity in both animal models (Amann, et al., Pain 64,323-329 (1996); Andreev, et al., Pain 63, 109-115 (1995)) and human (Dyck, et al., Neurology 48, 501-505 (1997); Petty, et al., Annals Neurol 36, 244-246 (1994)). NGF acts directly to induce pain in humans since it has been found that removal of the receptors for NGF leads to decreased pain sensation (Indo, Hum Mutat. 18(6), 462-71 (2001); Miura, et al., Hum Genet. 106(1), 116-24 (2000)), while pain is associated with an increase in NGF receptor expression in the spinal cord (Pezet, et al., J Neurosci. 19(13), 5482-5492) (1999) in humans.

In addition to its effects in the nervous system, NGF has been increasingly implicated in processes outside of the nervous system. For example, NGF has been shown to enhance vascular permeability (Otten, et al., Eur J Pharmacol. 106:199-201 (1984)), enhance T- and B-cell immune responses (Otten, et al., Proc. Natl. Acad. Sci. U.S.A. 86:10059-10063 (1989)), induce lymphocyte differentiation and mast cell proliferation and cause the release of soluble biological signals from mast cells (Matsuda, et al., Proc. Natl. Acad. Sci. U.S.A. 85:6508-6512 (1988); Pearce, et al., J. Physiol. 372:379-393 (1986); Bischoff, et al., Blood 79:2662-2669 (1992); Horigome, et al., J. Biol. Chem. 268:14881-14887 (1993)).

NGF is produced by a number of cell types including mast cells (Leon, et al, Proc. Natl. Acad. Sci. USA. 91:3739-3743 (1994)), B-lymphocytes (Torcia, et al., Cell 85:345-356 (1996), keratinocytes (Di Marco, et al., J. Biol. Chem. 268:22838-22846)) and smooth muscle cells (Ueyama, et al, J. Hypertens. 11:1061-1065 (1993)). NGF receptors have been found on a variety of cell types outside of the nervous system. For example, TrkA has been found on human monocytes, T- and B-lymphocytes and mast cells.

An association between increased NGF levels and a variety of inflammatory conditions has been observed in human patients as well as in several animal models. These include systemic lupus erythematosus (Bracci-Laudiero, et al., Neuroreport 4:563-565 (1993)), multiple sclerosis (Bracci-Laudiero, et al., Neurosci_{—}Lett. 147:9-12 (1992)), psoriasis (Raychaudhuri, et al., Acta Derm. l'enereol. 78:84-86 (1998)), arthritis (Falcim, et al., Ann. Rheum. Dis. 55:745-748 (1996)), asthma (Braun, et al., Eur. J Immunol. 28:3240-3251 (1998)), allergic inflammation (Bonini, et al., Proc. Natl. Acad. Sci. U.S.A. 93:10955-10960 (1996)).

Given the large number of biological activities or processes in which NGF plays a role, there is substantial interest in identifying agents capable of modulating NGF activity. The process of identifying such agents is primarily limited by the absence of informative screening assays. For a screening assay to be informative, it should evaluate the candidate agent's therapeutic potential by evaluating more than one endpoint, recapitulate the in vivo mechanism of action, capture compounds that inhibit the interactions between NGF and it receptor, downstream events in the NGF signal transduction pathway, as well as independent signaling pathways that inhibit the action of NGF indirectly. There is, therefore, great interest in identifying new assays for evaluating the therapeutic potential of modulators of NGF activity. This invention provides such an assay.

### SUMMARY OF THE INVENTION

The invention relates, in general, to a method of screening for agents that modulate NGF activity, specifically this invention provides a high throughput, cost effective, efficient, and sensitive screening method for assessing the ability of a candidate agent to modulate NGF activity. More specifically this invention provides a method of assessing the ability of a candidate agent to modulate (e.g., antagonize) NGF activity comprising, (a) contacting a culture of neurons expressing the high-affinity trk A receptor with a candidate agent and NGF and (b) measuring the level of expression of one or more NGF responsive genes, preferably at least two NGF responsive genes, in said culture, wherein an alteration of the level of expression of the one or more genes indicates the therapeutic potential of the candidate agent for treating symptoms associated with NGF activity in a subject and/or the dosage range that may be used in in vivo therapy. In addition, this invention provides a method of culturing neurons expressing high-affinity trk A receptor primary neurons, such as nociceptive neurons (e.g., dorsal root ganglion) for use in the methods described herein and methods of isolating polynucleotides (e.g., RNA) from such cultures.

One aspect of the invention provides a method of assessing the ability of a candidate agent to modulate NGF activity comprising: (a) contacting a culture of neurons expressing the high-anity trk A receptor with a candidate agent and NGF, and (b) measuring the level of expression of one or more NGF responsive genes in said culture, wherein an alteration of the level of expression of the one or more genes relative to an untreated culture of neurons indicates the therapeutic potential of the candidate agent for treating symptoms associated with NGF expression.

Another aspect of the invention provides a method of assessing the therapeutic potential of a candidate agent to modulate NGF activity comprising: (a) contacting a culture of neurons expressing the high-affinity trk A receptor with a candidate agent and NGF, and (b) measuring the level of expression of two or more NGF responsive genes in the culture, wherein an alteration of the level of expression of the two or more genes relative to an untreated nociceptive neuronal culture indicates the therapeutic potential of the candidate agent for treating symptoms associated with NGF expression.

In another aspect, the invention provides a method of assessing the therapeutic potential of a candidate agent to modulate NGF activity, wherein the culture of neurons expressing the high-affinity trk A receptor is a primary nociceptive neuronal culture (e.g., primary culture of dorsal root ganglion (DRG) neurons).

In yet another aspect, this invention provides a method of assessing the therapeutic potential of a candidate agent to modulate NGF activity, over a broad concentration range of NGF (e.g., between about 0.1 ng/ml to about 50 ng/ml).

Another aspect of this invention provides a method of assessing the therapeutic potential of a candidate agent to modulate NGF activity, utilzing a low number of cells (e.g., between about 100 to about 200 cells per well in a 96 well plate or between about 3.5 cells per square millimeter (mm²) to about 7 cells per square millimeter).

Yet another aspect the invention provides a method of assessing the therapeutic potential of a candidate agent to antagonize NGF activity.

Yet another aspect of the invention provides a method of assessing the therapeutic potential of a candidate agent to antagonize NGF activity, wherein the expression of at least one of the one or more genes is enhanced by NGF and/or the expression of at least one of the one or more genes is diminished by NGF or combinations thereof.

In yet another aspect, the invention provides a method of assessing the therapeutic potential of a candidate agent to inhibit NGF activity, wherein the level of expression of at least one of the two or more genes is enhanced by NGF and the expression of at least one of the two or more genes is diminished by NGF.

In yet another aspect, the invention provides a method of assessing the therapeutic potential of a candidate agent to antagonize NGF activity (i.e., an NGF antagonist), wherein an alteration in the level of expression of the one or more NGF responsive genes relative to an untreated nociceptive neuronal culture indicates the therapeutic potential of the candidate agent for treating symptoms associated with NGF expression and wherein the one or more genes are substance P and/or galanin or substance P and/or sprr1A.

In another aspect, the invention provides a method of assessing the therapeutic potential of a candidate agent to inhibit NGF activity, wherein an alteration in the level of expression of the one or more NGF responsive genes relative to an untreated nociceptive neuronal culture indicates the therapeutic potential of the candidate agent for treating symptoms associated with NGF expression and wherein the one or more genes are substance P and/or galanin.

Yet another aspect of this invention provides a method of cultuing neurons expressing high-affinity trk A receptor primary neurons, such as nociceptive neurons (e.g., dorsal root ganglion) for use in the methods described herein and methods of maximizing isolation and/or recovery of polynucleotides (e.g., RNA) from such cultures.

Another aspect of the invention provides a method of assessing the therapeutic potential of a combination therapy which possess synergistic effects leading to superior therapeutic regimens. This method comprises: (a) contacting a culture of neurons expressing the high-affinity trk A receptor with two or more candidate agents and NGF; and (b) measuring the level of expression of at least two NGF responsive genes in said culture, wherein an alteration of the level of expression of the genes relative to an untreated neuronal culture indicates the therapeutic potential of the candidate agent for treating symptoms associated with NGF expression.

Another aspect of the invention provides a method of assessing the therapeutic potential of a combination therapy to modulate NGF activity, wherein the culture of neurons expressing the high-affinity trk A receptor is a primary nociceptive neuronal culture (e.g., primary culture of dorsal root ganglion (DRG) neurons).

In yet another aspect the invention provides a method of assessing the therapeutic potential of a combination therapy to antagonize NGF activity, wherein the primary neuronal culture is a primary nociceptive neuronal culture (e.g., primary culture of dorsal root ganglion (DRG) neurons).

In another aspect, this invention provides a method of assessing the therapeutic potential of combination therapies to modulate NGF activity, over a broad concentration range of NGF (e.g., between about 0.1 ng/ml to about 50 ng/ml).

Another aspect of this invention provides a method of assessing the therapeutic potential of a candidate agent to modulate NGF activity, utilizing a low number of cells (e.g., between about 100 to about 200 cells per well in a 96 well plate or between about 3.5 cells per square millimeter (mm²) to about 7 cells per square millimeter).

### BRIEF DESCRIPTION OF THE DRAWING

FIG. 1. Graph of the fold difference (relative to cultures containing no added NGF) in the levels of substance P mRNA relative to GAPDH mRNA, galanin mRNA relative to GAPDH mRNA and substance P mRNA relative to galanin mRNA over a range of NGF concentrations up to a maximum of 50 ng/ml.

### DETAILED DESCRIPTION OF THE INVENTION

Throughout the disclosure, various publications, patents and published patent specifications are referenced by an identifying citation. The disclosure of these publications, patents and published patent specifications are hereby incorporated by reference into the present disclosure to more fully describe the state of the art to which this invention pertains.

Definitions

The practice of the present invention will employ, unless otherwise indicated, conventional techniques of immunology, molecular biology, microbiology, cell biology and recombinant DNA. Such techniques are explained fully in the literature, such as, for example, Sambrook, et al. MOLECULAR CLONING: A LABORATORY MANUAL, 2^{nd} edition (1989); CURRENT PROTOCOLS IN MOLECULAR BIOLOGY (F. M. Ausubel, et al. eds., (1987); the series METHODS IN ENZYMOLOGY (Academic Press, Inc.); “PCR: A PRACTICAL APPROACH” (M. MacPherson, et al., IRL Press at Oxford University Press (1991); PCR 2: A PRACTICAL APPROACH (M. J. MacPherson, B. D. Hames and G. R. Taylor eds (1995)); ANTIBODIES, A LABORATORY MANUAL (Harlow and Lane, eds (1988)); and ANIMAL CELL CULTURE (R. I. Freshney, ed. (1987)).

As used in the specification and claims, the singular form “a”, “an” and “the” include plural references unless the context clearly dictates otherwise. For example, the term “a gene” includes a plurality of genes, including mixtures thereof.

The term “polynucleotide” refers to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules.

The term “gene” refers to a polynucleotide containing at least one open reading frame that is capable of encoding a particular polypeptide or protein after being transcribed and translated.

The term “gene product” refers to the amino acid (e.g., peptide or polypeptide) generated when a gene is transcribed and translated.

The term “polypeptide” refers to polymeric forms of amino acids of any length.

The term “expression” includes production of a gene transcript and/or polypeptide.

As used herein, the term “nerve growth factor” and “NGF” refers to nerve growth factor and variants thereof that retain at least part of the activity of NGF. As used herein, NGF includes all mammalian species of native sequence NGF, including human, canine, feline, equine, or bovine.

“NGF activity” generally refers to the ability to bind NGF receptors and/or activate NGF receptor signaling pathways. Without limitation, a biological activity includes any one or more of the following: the ability to bind an NGF receptor (such as p75 and/or trkA); the ability to inhibit trkA receptor dimerization and/or autophosphorylation; the ability to activate an NGF receptor signaling pathway; the ability to promote cell differentiation, proliferation, survival, growth and other changes in cell physiology, including (in the case of neurons, including peripheral and central neuron) change in neuronal morphology, synaptogenesis, synaptic function, neurotransmitter and/or neuropeptide release and regeneration following damage; and the ability to mediate pain.

“NGF receptor” refers to a polypeptide that is bound by or activated by NGF. NGF receptors include the TrkA receptor and the p75 receptor of any mammalian species, including, but are not limited to, human, canine, feline, equine, primate, or bovine.

As used herein, the term “modulate” refers to an alteration or modification in the activity of NGF such as, for example, downstream pathways mediated by NGF signaling, such as receptor binding and/or elicitation of a cellular response to NGF. Examples of such alteration or modification may include, but is not limited to, enhancement or diminishment of NGF activity, enhancement or diminishment of symptoms associated with NGF activity and/or, enhancement or diminishment of gene expression.

An “NGF antagonist” refers to any molecule that blocks, suppresses or reduces (including significantly) NGF biological activity, including downstream pathways mediated by NGF signaling, such as receptor binding and/or elicitation of a cellular response to NGF and/or of gene expression.

The term “antagonist” implies no specific mechanism of biological action whatsoever, and is deemed to expressly include and encompass all possible pharmacological, physiological, and biochemical interactions with NGF whether direct or indirect, or whether interacting with NGF, its receptor, or through another mechanism, and its consequences which can be achieved by a variety of different, and chemically divergent, compositions. Exemplary NGF antagonists include, but are not limited to, an anti-NGF antibody, an anti-sense molecule directed to an NGF or NGF receptor (including an anti-sense molecule directed to a nucleic acid encoding NGF, trkA and/or p75), an NGF inhibitory compound, an NGF structural analog, a dominant-negative mutation of a TrkA receptor that binds an NGF, a TrkA immunoadhesin, an anti-TrkA antibody, an anti-p75 antibody, and a kinase inhibitor. For purpose of the present invention, it will be explicitly understood that the term “antagonist” encompass all the previously identified terms, titles, and functional states and characteristics whereby the NGF itself, an NGF biological activity (including but not limited to its ability to mediate any aspect of pain), or the consequences of the biological activity, are substantially nullified, decreased, or neutralized in any meaningful degree. In some embodiments, an NGF antagonist binds (physically interact with) NGF (e.g., an antibody), binds to an NGF receptor (such as trkA receptor or p75 receptor), reduces (impedes and/or blocks) downstream NGF receptor signaling, and/or inhibits (reduces) NGF synthesis, production or release. In some embodiments, an NGF antagonist binds (physically interacts with) NGF (e.g., an antibody), binds to an NGF receptor (such as trkA receptor or p75 receptor), and/or reduces (impedes and/or blocks) downstream NGF receptor signaling. In other embodiments, an NGF antagonist binds NGF and prevents trkA receptor dimerization and/or trkA autophosphorylation. In other embodiments, an NGF antagonist inhibits or reduces NGF and/or NGf receptor synthesis and/or production or expression. Examples of types of NGF antagonists are provided herein.

An “antibody” (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)2, Fv), single chain (ScFv), mutants thereof, fusion proteins comprising an antibody portion, humanized antibodies, chimeric antibodies, diabodies linear antibodies, single chain antibodies, multispecific antibodies (e.g., bispecific antibodies) and any other modified configuration of the immunoglobulin molecule that comprises an antigen recognition site of the required specificity. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A “monoclonal antibody” refers to a homogeneous antibody population wherein the monoclonal antibody is comprised of amino acids (naturally occurring and non-naturally occurring) that are involved in the selective binding of an antigen. Monoclonal antibodies are highly specific, being directed against a single antigenic site. The term “monoclonal antibody” encompasses not only intact monoclonal antibodies and full-length monoclonal antibodies, but also fragments thereof (such as Fab, Fab′, F(ab′)2, Fv), single chain (ScFv), mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.).

“Humanized” antibodies refer to a molecule having an antigen binding site that is substantially derived from an immunoglobulin from a non-human species and the remaining immunoglobulin structure of the molecule based upon the structure and/or sequence of a human immunoglobulin. The antigen binding site may comprise either complete variable domains fused onto constant domains or only the complementarity determining regions (CDRS) grafted onto appropriate framework regions in the variable domains. Antigen binding sites may be wild type or modified by one or more amino acid substitutions, e.g., modified to resemble human immunoglobulin more closely. Some forms of humanized antibodies preserve all CDR sequences (for example, a humanized mouse antibody which contains all six CDRs from the mouse antibodies). Other forms of humanized antibodies have one or more CDRs (one, two, three, four, five, six) which are altered with respect to the original antibody. In some instances, framework region (FR) residues or other residues of the human immunoglobulin replaced by corresponding non-human residues. Furthermore, humanized antibodies may comprise residues which are not found in the recipient antibody or in the donor antibody.

As used herein, an “anti-NGF antibody” refers to an antibody which is able to bind to NGF and inhibit NGF biological activity and/or downstream pathway(s) mediated by NGF signaling.

A “TrkA immunoadhesin” refers to a soluble chimeric molecule comprising a fragment of a TrkA receptor, for example, the extracellular domain of a TrkA receptor and an immunoglobulin sequence, which retains the binding specificity of the TrkA receptor.

The term “epitope” is used to refer to binding sites for (monoclonal or polyclonal) antibodies on protein antigens.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as DNA polymerase, and typically a thermally-stable polymerase enzyme. Methods for PCR are well known in the art, and taught, for example in “PCR: A PRACTICAL APPROACH” (M. MacPherson et al., IRL Press at Oxford University Press (1991)). All processes of producing replicate copies of a polynucleotide, such as PCR or gene cloning, are collectively referred to herein a “replication.” A primer can also be used as a probe in hybridization reactions, such as Southern or Northern blot analyses. Sambrook et al., supra.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For example of carriers, stabilizers and adjuvants, see Martin, REMINGTON'S PHARM. SCI., 15th Ed. (Mack Publ. Co., Easton (1975)).

An “effective amount” is an amount sufficient to effect beneficial or desired results. An effective amount can be administered in one or more administrations, applications or dosages.

A “subject,” “individual” or “patient” is used interchangeably herein, which refers to a vertebrate, preferably a mammal, more preferably a human. Mammals include, but are not limited to, murines, simians, humans, farm animals, sport animals, and pets. As used herein, “teatment” is an approach for obtaining beneficial or desired clinical results. For purposes of this invention, beneficial or desired clinical results include, but are not limited to, one or more of the following: improvement or alleviation of any aspect of pain, including acute, chronic, inflammatory, neuropathic, or post-surgical pain. For purposes of this invention, beneficial or desired clinical results include, but are not limited to, one or more of the following: including lessening severity, alleviation of one or more symptoms associated with pain including any aspect of pain (such as shortening duration of pain, and/or reduction of pain sensitivity or sensation).

The term “culture” or “culturing” refers to in vitro maintenance and/or propagation of cells and/or organisms on or in media of various kinds.

The term “medium” or “media” refers to the aqueous environment, which provides, for example, the physicochemical, nutritional, and hormonal requirements for cell survival in culture. Serum-free medium” refers to a medium lacking serum. The hormone, growth factors, transport proteins, peptide hormones and the like typically found in serum which are necessary for the survival or growth of particular cells in culture are typically added as a supplement to serum-free medium. A “defined medium” refers to a medium comprising nutritional and hormonal requirements necessary for the survival and growth of the cells in culture such that the components of the medium are known.

Methods

The invention relates, in general, to a method of screening for agents that modulate NGF activity and methods of culturing primary neuronal cultures for use in such methods. In particular this invention provides a high throughput, cost effective, efficient, and sensitive screening method for assessing the ability of a candidate agent to modulate NGF activity. This invention is based on a discovery that gene expression in nociceptive neurons is a sensitive and informative assay for evaluating candidate agents for treating NGF associated diseases or conditions. This invention provides several advantages or improvements, including, but not limited to, the use of a physiologically relevant population of neurons (e.g., a primary culture), high throughput capability (e.g., use of low density number of cells, for example a density of about 100 to about 200 cells per well in a 96 well plate or between about 3.5 cells per square millimeter (mm²) to about 7 cells per square millimeter), enhanced extraction and detection of polynucleotides (e.g., enhanced RNA recovery from low cell density and detection by RT-PCR), broad dosage range of NGF for assay (e.g., between about 0.1 ng/ml to about 50 ng/ml), and the ability to assess the level of expression of gene combinations (e.g., substance P and galanin and/or Substance P and sprr1A) which facilitate detection of a broader range of moduators (e.g., partial antagonists) over a broader dosage range of NGF.

This invention generally provides a method of assessing the ability of a candidate agent to modulate NGF activity comprising, (a) contacting a culture of neurons expressing the high-affinity trk A receptor with a candidate agent and NGF, and (b) measuring the level of expression of one or more NGF responsive genes (e.g., two genes) in said culture, wherein an alteration of the level of expression of the genes indicates the therapeutic potential of the candidate agent for treating symptoms associated with NGF activity in a subject and/or the dosage range that may be used in in vivo therapy.

In some embodiments the culture of neurons expressing the high-affinity trk A receptor is a primary nociceptive neuronal culture, such as, by way of example, a primary culture of dorsal root ganglion (DRG) neurons. In a preferred embodiment the primary nociceptive neuronal culture is maintained in culture by the methods described herein below.

In other embodiments the method of assessing the therapeutic potential of a candidate agent to modulate NGF activity is evaluated by measuring the level of expression of two or more NGF responsive genes. By way of example, the two or more genes may comprise substance P, galanin or sspr1A. In a preferred embodiment, the level of expression of seubstance P and galanin or substance P and sspr1A is used in the methods described herein to evaluate the ability of a candidate agent to antagonize NGF activity.

In other embodiments the methods described herein are used to assess the therapeutic potential of a combination therapy which possess synergistic effects leading to superior therapeutic regimens or other combination therapies.

Neuronal Cultures

The neuronal cultures preferably comprise neurons expressing the high-affinity trkA receptor to which NGF binds. In a preferred embodiment, the neurons selected for culture are neurons which exhibit a change in their pattern of gene expression in response to NGF (e.g., NGF responsive), but do not require NGF for survival. In a preferred embodiment the neuronal cultures are primary cultures of neurons expressing the high-affinity trkA receptor to which NGF binds. Examples of neurons include, but are not limited to, nociceptive neurons of the DRG, nociceptive and non nociceptive trigeminal ganglion neurons, sympathetic neurons, NGF responsive subpopulations of the nodose ganglia and basal forebrain cholinergic neurons. Non limiting examples of nociceptive neurons include, but are not limited to, DRG neurons and nociceptive neurons within the trigeminal ganglion. The neurons used in the cultures may be isolated from a variety of animals, including, but not limited to, primates, rodents such as adult rats or mice. In a preferred embodiment DRG neurons from adult rats may be utilized.

Evidence from in situ hybridization for trkA mRNA (Carroll et al., 1992 Neuron 9:779-788; Mu et al., 1993 J. neuroscience 13:4029-4041; McMahon et al., 1994 Neuron 12:1161-1171; Wright and Snider, 1995 J. Comp Neurol 351:329-338), high-affinity binding of labeled NGF (Verge et al., 1992 J. Neurosci 12: 4011-4022; Richardson and Verge et al., 1986 J. Neurocyt. 15:585-594; Verge et al., 1989 J. Neurocyt. 18: 583-591; Verge et al. 1989 J. Neurosci 9:914-922; Verge et al., J. Neurosci 10:2018-2025), retrograde transport of iodinated NGF from peripheral nerve to DRG (Richardson et al., 1984 J. Neroscience 4: 1683-1689; DiStefano et al., 1992 Neuron 8:983-993) and immunohistochemistry (DiStefano et al., 1992), indicate that 45% of lumbar DRG are small diameter, trkA-expressing nociceptive neurons. Adult DRG neurons survive in culture independently of NGF (Lindsay, 1988).

DRG neurons may be isolated from a variety of animals, including, but not limited to primates or rodents. Examples of rodents include, but are not limited to, adult rats or adult mice. DRG neurons may be harvested/isolated by conventional methodology. By way of example, dorsal root ganglia were dissected from adult (3-6 months old) Sprague-Dawley rats.

For primary cultures, generally prior to culturing the isolated cells are subjected to a preincubation step. The preincubation step preferably involves contacting the isolated cells with enzymes such as one or more protease enzymes (e.g., collagenase or trypsin) to reduce the level of, for example, connective tissue and to facilitate dissociation of cells within the ganglia to a single cell suspension in culture. One of skill in the art will appreciate that the period for preincubation will vary depending on the protease utilized and the condition of the isolated cells, but may be, for example, for a total period of between about 1 to about 4 hours. Excess enzyme or other agents utilized in the preincubation step can be removed by washing with culture media By way of example, dorsal root ganglia dissected from adult (3-6 months old) Sprague-Dawley rats may be stripped of their sheaths, and incubated in 0.125% collagenase type IV (Worthington) in Ham's F12 with 10% HIHS for two 90 minute periods at 37° C. They may then be washed extensively and incubated in 0.25% trypsin (Worthington) in HBSS at 37 degrees for 30 minutes. After inactivation of trypsin with 10% heat inactivated horse serum and further washing, the ganglia may be dissociated by gentle trituration through a flame polished Pasteur pipette.

The isolated cells may be subjected to the preincubation step and/or subjected directly to culturing procedure using conventional methodology (e.g., Lindsay 1988). Various cell culture systems are known to the ordinarily skilled artisan. The culture platform or solid matrix (e.g. multiwell plate, or plastic flask culture dish or plate) is coated with extracellular matrix/adhesion proteins such as for example, polyornithine, laminin, fibronectin, poly-lysine or collagen. In a preferred embodiment the platform is coated with polyornithine and laminin. Suitable culture media are also known to persons skilled in the art and include, but are not limited to, commercially available media such as Ham's F14, Minimal Essential Medium (MEM), RPMI-1640, and Dulbecco's Modified Eagle's Medium (DMEM). The media can be supplemented with mitogenic agents, ions (such as sodium, chloride, calcium, magnesium, and phosphate), buffers, nucleosides, trace elements, antibiotics, glucose or an equivalent energy source, salts of metals, amino acids hormones and proteins. Appropriate concentrations are known to those skilled in the art.

Additional reagents may also be added to the media without effecting the sensitivity or responsiveness (e.g., gene expression) of the screening method. Candidate agents to be screened by the methods described herein may be dissolved in an organic solvent such as, but not limited to dimethyl sulphoxide (DMSO). By way of example, concentrations of lower than or equal to about 1% DMSO, such as about 0.1% may be present in the media without effecting the sensitivity or responsiveness.

In a preferred embodiment, a primary culture of DRG neurons, the neurons are grown in serum free medium and plated on polyornithine and laminin coated 96-well plates. Defined medium consisting of Hams F-14 nutrient mixture with plus 2 mM glutamine, 0.35% bovine serum albumin (Albumax II, Gibco-BRL or Pathocyte-5, ICN), 60 ng/ml progesterone, 16 ug/ml putrescine, 400 ng/ml L-thyroxine, 38 ng/ml sodium selenite, 340 ng/ml tri-iodo-thyronine, 60 ug/ml penicillin and 100 ug/ml streptomycin (Sigma) (Davies et al., 1993). NGF (10 ng/ml) and test compounds were added to the wells shortly before or after plating.

The plating density of the primary culture is determined by a variety of parameters including, but not limited to, signal to noise ratio, sensitivity of RNA detection method reaction (e.g., RT-PCR) and basal level of expression of the one or more genes (e.g., target gene expression) in the assay. By way of example, for a 96 well plate cells, such as DRG, may be plated at between about 100 to about 1000 cells per well (e.g., between about 3.5 cells per square millimeter to about 35 cells per square millimeter) or between about 100 cells or about 200 cells per well (e.g., between about 3.5 cells per square millimeter to about 7 cells per square millimeter). In a preferred embodiment the cells are plated at about 200 cells per well. By way of example if the target gene expression is substance P and galanin or substance P and sspr1A, the cell density is about 200 cells per well.

Generally to evaluate the therapeutic potential of a candidate agent the cells in culture are contacted with a concentration range of NGF. For example, cells may be contacted or incubated with between about 0.01 ng/ml to about 1000 ng/ml, such as at about 0.5 ng/ml to about 50 ng/ml, or such as at about 0.1 ng/ml to about 10 ng/ml of NGF. Preferably there are replicate platings for each cell density and/or each different concentration of NGF. The NGF utilized may be isolated or produced by methods known in the art The NGF may be added to the culture at any point. For example, the NGF may be added to the culture prior to the addition of the candidate agent, concurrently with the candidate agent or after the addition of the candidate agent. Cells in culture may be incubated with NGF for a variety of times. For example, the cells may be incubated for between about 24 hours to between about 120 hours. By way of example, NGF can be added concurrently with the candidate agent and incubated for about 120 hours. Parameters to be considered in determining the time period for incubation include, but are not limited to, the time period at which the ratio between the expression of two or more genes (e.g., substance P and galanin or substance P and sspr1A) is highest and/or the time period for at which the levels of the mRNAs for the two or more genes is sufficient in the absence of NGF to be detected by, for example, RT-PCR.

Genes

In the subject invention the expression of one or more genes, in response to NGF and a candidate agent is evaluated. By way of example, the expression of two or more genes is evaluated. In a preferred embodiment, the expression of one of the two genes is enhanced in the presence of NGF and the expression of one of the two genes is diminished in the presence of NGF.

Examples of genes whose expression is enhanced (e.g., NGF responsive) in the presence of NGF includes but is not limited to spinocerebellar ataxia type 1 (sca 1), substance P, lymphocyte antigen 86(MD-1), Hippocampus cDNA homologue to Microsomal Signal Peptidase, Neuronal Leucine Rich Repeat Protein 1 (NLRR-1), Synaptotagnin V, Cadherin 1, ESTs weakly similar to KIAA0982 protein and RIKEN cDNA 2310042NO2 gene. Examples of genes whose expression is diminished (e.g., NGF responsive) in the presence of NGF includes, but is not limited to small proline-rich repeat protein 1A, Motopsin (Neurotrypsin), Inhibin Beta B, G protein-coupled receptor 19, Lipocalin 2, and Troponin C. By way of example, in one assay Substance P and galanin and/or small proline rich repeat protein 1A (sprr1A) can be used. The assay may further comprise evaluating the expression of one or more genes as a control (e.g., genes whose expression is not altered by NGF, such as a housekeeping gene). Examples of such genes include, but are not limited to, GAPDH, 18S, 28S and L27 ribosomal RNA. Such genes can also serve as an indicator of cell viability. If the level of expression of two or more genes is evaluated, preferred combinations include, but are not limited to substance P and galanin or substance P and sspr1A.

Isolating Polynucleotides and Evaluating Gene Expression

Polynucleotides (e.g., DNA or RNA) can be isolated from cells by a variety of methods known in the art. Preferably, the isolation method can efficiently isolate RNA from a low density of cells (e.g., 100 to 200 neurons) to optimize detection of the RNA. In a preferred embodiment, silica based magnetic beads (Levinson et al., J. Chromatogr A. Aug. 7, 1998: 816(1):107-11), such as, for example, Magnasil (Promega), that bind both DNA and RNA under high salt conditions are utilized. The cells may be lysed by conventional methodolgy. By way of example, the cells in each well of a 96 well plate are lysed in 100 ul of 4M guanidine hydrochloride containing 10 mm Tris (pH 7.5) and about 7.5 ul of magnasil can be added to each well and mixed thoroughly with the cell lysate. After about 10 minutes incubation at room temperature, the beads are pelleted to the side of the wells by placing the plate on a 96 well magnet. After aspirating the lysate with a pipette (e.g., multi channel pipette), about 150 ul of about 80% ethanol was added to each well and the plate removed from the magnet to allow the magnasil to be resuspended by pipetting up and down. The use of 80% ethanol is a preferred embodiment as the about 80% ethanol preferentially removes genomic DNA from the beads over RNA. Following than ethanol wash, the magnasil can be washed a further two times with about 150 ul of about 80% ethanol, before being allowed to air dry. RNA is eluted from the air dried magnasil by the addition of nuclease free water (e.g., 75 ul). Preferably primers used in the detection method described herein below are intron spanning primers thereby decreasing amplification of for example, genomic DNA. In contrast to conventional RNA isolation methods, this method when used in conjunction with intron spanning primers, does not require the addition of DNAse for the removal of genomic DNA to maintain the accuracy and sensitivity of the assay even if intronless pseudogenes corresponding to the target genes exist and/or introns between the PCR primers are small. This improvement thereby provides for cost effective high throughput screening.

Gene expression can be evaluated by methods known in the art. Methods for isolating RNA and/or protein from cells and/or detection/quanitation of RNA and protein for evaluation are known in the art. By way of example, the cells may be lysed and RNA extracted utilizing the guanidine hydrochloride method and RNA analyzed using multiplexed Real Time-PCR (Wittwer et al (2001) Methods 24:430-442). In a particularly preferred embodiment molecular beacon probes are used in the methods described herein (Tyagi et al (1998) Nature Biotechnology 16:49-53; Broude et al (2002) Trends in Biotechnology 20(6):249-56).

Examples of primers, such as for PCR, for substance P, include, but are not limited to, the forward primer sequence:5′-GAGGAAATCGGTGCCAACG-3′ and the reverse primer sequence: 5′-TCTCTGAAGAAGATGCTCAAAGG-3′. The sequence of the substance P Molecular Beacon probe may be, for example, 5′-CGCGATGTCGGACCAGTCGGACCAATTCGCG-3′. Examples of primers, such as for PCR, for GAPDH include, but are not limited to, the forward primer :−5′-TATCGGACGCCTGGTTAC-3′ and the reverse primer 5′-AACTTGCCGTGGGTAGAG-3′ The molecular beacon probe for GAPDH can be, for example, 5′-CGCGATCAAGTGGACATTGTTGCCATCAACGACGATCGCG-3′. Examples of primers, such as for PCR, for galanin include, but are not limited to, 5′-CCCACATGCCATTGACAACC-3′ and the reverse primer is 5′-CGGACGATATTGCTCTCAGG-3′. By way of example, for the reaction, the annealing temperature may be between about 55° C. to about 62° C. and the magnesium concentration may be between about 2 millimolar (mm) to about 6 mM (e.g., 2 mM, 3 mM, 4 mM, 5 mM or 6 mM).

Candidate Agents

Candidate agents suitable for assaying in the methods of the subject application may be any type of molecule from, for example, chemical, nutritional or biological sources. The agent may be naturally occurring or synthetically produced. For example, the agent may encompass numerous chemical classes, though typically they are organic molecules, preferably small organic compounds having a molecular weight of more than 50 and less than about 2,500 Daltons. Such molecules may comprise functional groups nnecessary for structural interaction with proteins or nucleic acids. By way of example, chemical agents may be novel, untested chemicals, agonists, antagonists, or modifications of known therapeutic agents.

The agents may also be found among biomolecules including, but not limited to, peptides, saccharides, fatty acids, antibodies, steroids, purines pryimidines, derivatives or structural analogs thereof or amolecule manufactured to mimic the effect of a biological response modifier. Examples of agents from nutritional sources include, but is not limited to, extracts from plant or animal sources or extracts thereof.

Agents may be obtained from a may be obtained from a wide variety of sources including libraries of synthetic or natural compounds. Alternatively, libraries of natural compounds in the form of bacterial, fungal, plant, and animal extracts are available or readily produced. Additionally, natural or synthetically produced libraries or compounds are readily modified through conventional chemical, physical and biochemical means, and may be used to produce combinatorial libraries. Known pharmacological agents may be subjected to random or directed chemical modifications, such as acylation, alkylation, esterification, amidification, etc. to produce structural analogs. Preferred candidate agents are NGF antagonists.

In the methods described herein for evaluating a combination therapy which possess synergistic effects leading to superior therapeutic regimens or combination therapies the one or more agents comprising the combination may be novel or modifications of known therapeutic agents. By way of example, the combination may comprise a NGF antagonist and an opioid analgesic.

NGF Antagonists

The methods of this invention can screen for an NGF antagonist, which refers to any molecule that blocks, suppresses or reduces (including significantly) NGF biological activity, including downstream pathways mediated by NGF signaling, such as receptor binding and/or elicitation of a cellular response to NGF. The term “antagonist” implies no specific mechanism of biological action whatsoever, and is deemed to expressly include and encompass all possible pharmacological, physiological, and biochemical interactions with NGF and its consequences which can be achieved by a variety of different, and chemically divergent, compositions. Exemplary types of agents that may be screened for ability to antagonize NGF activity include, but are not limited to, an anti-NGF antibody, an anti-sense molecule directed to an NGF (including an anti-sense molecule directed to a nucleic acid encoding NGF), an NGF inhibitory compound, an NGF structural analog, a dominant-negative mutation of a TrkA receptor that binds an NGF, a TrkA immunoadhesin, an anti-TrkA antibody, an anti-p75 antibody, a kinase inhibitor and small molecules having a molecular weight of 100 to 20,000 daltons, 500 to 15,000 daltons, or 1000 to 10,000 daltons. Libraries of small molecules are commercially available.

For purpose of the present invention, it will be explicitly understood that the term “antagonist” encompass all the previously identified terms, titles, and functional states and characteristics whereby the NGF itself, an NGF biological activity (including but not limited to its ability to ability to mediate any aspect of pain), or the consequences of the biological activity, are substantially nullified, decreased, or neutralized in any meaningful degree. In some embodiments, an NGF antagonist binds (physically interact with) NGF (e.g., an antibody), binds to an NGF receptor (such as trkA receptor or p75 receptor), and/or reduces (impedes and/or blocks) downstream NGF receptor signaling.

Anti-NGF Antibodies

In a preferred embodiment the candidate antagonist is an anti-NGF antibody. The antibodies can be monoclonal antibodies, polyclonal antibodies, antibody fragments (e.g., Fab, Fab′, F(ab′)2, Fv, Fc, etc.), chimeric antibodies, bispecific antibodies, heteroconjugate antibodies, single chain (ScFv), mutants thereof, fusion proteins comprising an antibody portion, humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or any other origin (including chimeric or humanized antibodies). The epitope(s) can be continuous or discontinuous. In one aspect, antibodies (e.g., human, humanized, mouse, chimeric) that can inhibit NGF may be made by using immunogens that express full length or partial sequence of NGF. In another aspect, an immunogen comprising a cell that overexpresses NGF may be used. Another example of an immunogen that can be used is NGF protein that contains full-length NGF or a portion of the NGF protein. The anti-NGF antibodies may be made by any method known in the art and tested by the method described herein. In an alternative, antibodies may be made recombinantly and expressed using any method known in the art. In another alternative, antibodies may be made recombinantly by phage display technology. See, for example, U.S. Pat. Nos. 5,565,332; 5,580,717; 5,733,743; 6,265,150; and Winter et al., Annu. Rev. Immunol. 12:433-455 (1994). Alternatively, the phage display technology (McCafferty et al., Nature 348:552-553 (1990)) can be used to produce human antibodies and antibody fragments in vitro, from immunoglobulin variable (V) domain gene repertoires from unimmunized donors.

### THERAPEUTIC APPLICATIONS

The candidate agents identified by the methods described herein would have therapeutic applications for a variety of condition or diseases associated with NGF. The methods and compositions of the present invention are useful for the treatment of pain of any etiology, including acute and chronic pain, any pain with an inflammatory component, and any pain in which an opioid analgesic is usually prescribed. Examples of pain include post-operative pain (including dental pain), migraine, headache and trigeminal neuralgia, pain associated with burn, wound or kidney stone, pain associated with trauma (including traumatic head injury), neuropathic pain, pain associated with musculo-skeletal disorders such as rheumatoid arthritis, osteoarthritis, ankylosing spondylitis, sero-negative (non-rheumatoid) arthropathies, non-articular rheumatism and peri-articular disorders, and pain associated with cancer (including “break-through pain” and pain associated with terminal cancer), peripheral neuropathy and post-herpetic neuralgia. Examples of pain with an inflammatory component (in addition to some of those described above) include rheumatic pain, pain associated with mucositis, and dysmenorrhea. In some embodiments, the methods and compositions of the present invention are used for treatment or prevention of post-surgical pain and cancer pain.

NGF antagonists isolated by the methods described herein may also be used in the treatment of cardiac arrhythmia associated with abnormal levels of NGF, for autonomic dysreflexia, for asthma and for cancers whose proliferation, survival or metastasis is promoted by NGF (e.g., prostatic cancer and pancreatic cancer). NGF antagonists isolated by the methods described herein also may be used to treat cystitis, pancreatitis and sickle cell crisis.

The following Examples are provided to illustrate but not limit the invention.

### EXAMPLES

### Example 1

### Primary Rat DRG Neuronal Cultures

Dorsal root ganglia were dissected from adult (3-6 months old) Sprague-Dawley rats, and dissociated and cultured by standard techniques (Lindsay, 1988). Briefly, ganglia were stripped of their sheaths, and incubated in 0.125% collagenase type IV (Worthington) in Ham's F12 with 10% HIHS for two 90 minute periods at 37° C. They were then washed extensively and incubated in 0.25% trypsin (Worthington) in HBSS at 37 degrees for 30 minutes. After inactivation of trypsin with 10% heat inactivated horse serum and further washing, the ganglia were dissociated by gentle trituration through a flame polished Pasteur pipet.

Neurons were plated on polyornithine and laminin coated 96-well plates in defined serum free medium consisting of Hams F-14 nutrient mixture with plus 2 mM glutamine, 0.35% bovine serum albumin (Albumax II, Gibco-BRL or Pathocyte-5, ICN), 60 ng/ml progesterone, 16 ug/ml putrescine, 400 ng/ml L-thyroxine, 38 ng/ml sodium selenite, 340 ng/ml tri-iodo-thyronine, 60 ug/ml penicillin and 100 ug/ml streptomycin (Sigma) (Davies et al., 1993). NGF (10 ng/ml) and test compounds were added to the wells shortly before or after plating as described below.

### Example 2

### Establishment of a Gene Expression Based Assay for NGF Inhibitors

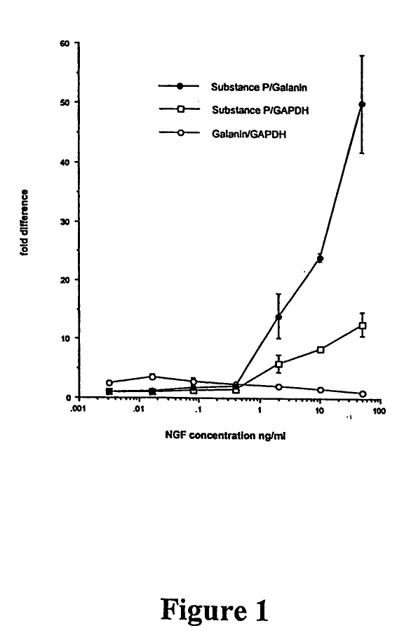

To identify genes that are either induced or suppressed by NGF, cDNA based gene arrays (Agilant Inc.) were utilized to identify NGF responsive genes in adult sensory neurons. Out of approximately 9,000 genes that were analyzed, nine genes that were markedly up-regulated by NGF and seven genes that were markedly down-regulated by NGF were identified (Table 1). Analysis by real time PCR revealed that of these genes, substance P (up-regulated by NGF) and galanin (down-regulated by NGF), would be optimal for a screen for NGF inhibitory compounds. Combining these two genes led to an assay that detects changes in the concentration of NGF of between 0.5 and 50 ng/ml and has a dynamic range of 50 fold (FIG. 1).

TABLE 1
Genes marked up-regulated and down-regulated by NGF in adult DRG
neurons cultured for 96 hours with NGF.

| UP-REGULATED GENES | Down-regulated genes |
|---|---|
| Spinocerebellar ataxia type 1 (Sca 1) | Small proline rich protein 1A |
| Substance P | Galanin |
| Lymphocyte antigen 86 (MD-1) | Motospin (Neurotrypsin) |
| Hippocampus cDNA homologue to | Inhibin beta B |
| Microsomal Signal Peptidase | G protein-coupled receptor 19 |
| Leucine rich reoeat protein 1, neuronal | Lipocalin 2 |
| (NLRR-1) | Troponin C |
| Synaptotagmin V | |
| Cadherin 1 | |
| RIKEN cDNA 2310042NO2 gene | |

### Example: 2

### Measurement of Gene Expression Expression in Adult DRG by Real-Time PCR

Adult rat DRG neurons were plated in 96 well plates, exposed to different concentrations of NGF for 120 hours and specific mRNA expression was analysed using the Stratagene Mx4000 real time PCR instrument. These conditions allow detection of greater than 15-fold increase in substance P mRNA content relative to GAPDH mRNA at the highest concentration of NGF used (FIG. 1). In addition, it was shown that a greater than 3-fold reduction in galanin mRNA level relative to GAPDH mRNA at the highest concentration of NGF can be measured (FIG. 1). When these genes are combined in a single assay the combined difference in gene expression in the absence of presence of NGF is approximately 50-fold suggesting that the assay would be sensitive and robust (FIG. 1).

After culture as described above for 5 days RNA was extracted and the ratio of substance P mRNA expression determined relative to GAPDH mRNA for RNA from each of the culture wells by RT-PCR. RT-PCR was also performed to determine the ratio of galanin to GAPDH mRNA for each RNA sample. Substance P and GAPDH were amplified or “multiplexed” in the same reaction using molecular beacon probes with two different flourophores to distinguish between the two different reaction products (Wittwer et al (2002) Methods 25:430-442). Galanin was amplified in a separate reaction.

The substance P forward primer sequence was: 5′-GAGGAAATCGGTGCCAACG-3′ and the reverse primer sequence was: 5′-TCTCTGAAGAAGATGCTCAAAGG-3′ The sequence of the substance P Molecular Beacon probe was 5′-CGCGATGTCGGACCAGTCGGACCAATTCGCG-3′. The 5′ end of the probe was labeled with FAM and the 3′ has a BHQ1 quencher attached. The GAPDH forward primer was: −5′-TATCGGACGCCTGGTTAC-3′ and the reverse primer was 5′-AACTTGCCGTGGGTAGAG-3′ The molecular beacon probe for GAPDH was 5′-CGCGATCAAGTGGACATTGTTGCCATCAACGACGATCGCG-3′ The 5′ end of the beacon is labeled with HEX and the 3′ end is quenched with a BHQ1 quencher

Following reverse transcription of RNA extracted from neuronal cultures, a proportion of the resulting cDNA was amplified in a reaction mixture containing both GAPDH and substance P primers and the substance P and GAPDH molecular beacon probes. The magnesium concentration in the reaction mix is 5 mM, the annealing temperature is 55 degrees C. for 1 min. Denaturation is 95 degrees for 1 minute and elongation is 72 degrees for 30 seconds 40 rounds of amplification are performed

The Galanin Forward primer was: −5′-CCCACATGCCATTGACAACC-3′ The Galanin reverse primer was 5′- CGGACGATATTGCTCTCAGG-3′. The annealing temperature for the galanin PCR reaction was 62 degrees C. and the magnesium concentration for the reaction was between 3-6 mM. Other cycling conditions are as above. Galanin PCR products were labeled by incorporation of Syber Gold and the identity of products confirmed post-PCR by melting point analysis.

### Example 3

### RNA Purification and Extraction

The assay with GAPDH, substance P and galanin does not require the removal of genomic DNA from the RNA prior to RT-PCR as all PCR reactions use intron spanning primers. Omitting the DNAse step increases the speed of the RNA extraction, reduces the cost and significantly increased the yield of RNA. The presence of genomic DNA does not influence the results of the assay.

In cases where mRNA levels of genes that do not allow the use of intron spanning primers are assayed, a DNAse digestion step is included in the protocol between the first and second ethanol washes. Primers spanning an intron were designed for GAPDH because the rat genome contains a number of GAPDH pseudogenes having the same sequence as the true GAPDH gene but containing no introns. For this reason contamination of RT-PCR reactions with signal resulting from genomic DNA was initially a problem when using the RNA extraction procedure that omitted the DNase step. This was also the case if the intron between the PCR primers is small. However, it was discovered that magnasil has higher affinity for RNA than double stranded genomic DNA.

In a series of experiments, the ethanol wash solutions were reduced stepwise from the original starting concentration of 95% ethanol to 75% ethanol. As the ethanol concentration falls there is a tendency for the nucleic acids to be removed from the magnasil into solution. Since the RNA has a higher affinity for magnasil than genomic DNA, the genomic DNA was eluted first. It was found that at 80% ethanol most of the genomic DNA comes off the beads and is removed in the wash solution while little RNA is lost.

In brief, cells were lysed in 100 ul of 4M gaunidine hydrochloride, 10 mm Tris (pH 7.5), 1% 2-mercaptoethanol. 7.5 u; of magnasil particles (Promega) were added to each RNA sample followed by mixing and incubation at room temp for 10 minutes with further frequent mixing. The magnetic particles were pelleted, by removing the cell culture plate to a magnet, and the lysis buffer was removed. The magnetic particles were washed three times with 150 ul of 80% ethanol before air drying them. RNA was eluted by the addition of 100 ul of nuclease free water to the dried beads.

In summary, the assay with the greatest sensitivity for identifying NGF inhibitors in adult rat DRGs were grown in 2 ng/ml NGF, at a density of 200 neurons per well, with no more than 0.5% DMSO, RNA was extracted after 5 days in culture and genomic DNA was not removed from the RNA.

Although the foregoing invention has been described in some detail by way of illustration and example for purposes of clarity of understanding, it will be apparent to those skilled in the art that certain changes and modifications may be practiced. Therefore, the descriptions and examples should not be construed as limiting the scope of the invention.

We claim:

1. A method of assessing the ability of a candidate agent to modulate NGF activity comprising:
(a) contacting a culture of neurons expressing the high-affinity trk A receptor with a candidate agent and Nerve Growth Factor, and
(b) measuring the level of expression of two or more NGF responsive genes in said culture, wherein an alteration of the level of expression of the two or more genes indicates the therapeutic potential of the candidate agent.

2. The method of claim 1, wherein the culture of neurons expressing the high-affinity trk A receptor is a primary neuronal culture.

3. The method of claim 2, wherein the neurons are nociceptive neurons of the Dorsal Root Ganglia, trigeminal ganglion nociceptive and non nociceptive neurons, sympathetic neurons or Nerve Growth Factor responsive subpopulations of the nodose ganglia and basal forebrain cholinergic neurons.

4. The method of claim 3, wherein the neurons are nociceptive neurons of the Dorsal Root Ganglia neurons.

5. The method of claim 1, wherein the neurons in the culture are between about 100- to about 1000 cells per well or between about 100- to about 200 cells per well.

6. The method of claim 5, wherein the neurons in the culture are between about between about 3.5 cells per square millimeter to about 35 cells per square millimeter or 3.5 cells per square millimeter to about 35 cells per square millimeter.

7. The method of claim 1, wherein the Nerve Growth Factor concentration is between about 0.01 ng/ml to about 1000 ng/ml.

8. The method of claim 7, wherein the Nerve Growth Factor concentration is between about 0.1 ng/ml to about 50 ng/ml.

9. The method of claim 1, wherein expression of at least one gene of the two or more genes is enhanced in the presence of Nerve Growth Factor.

10. The method of claim 9, wherein the gene is spinocerebellar ataxia type 1 (sca 1), substance P, lymphocyte antigen 86(MD-1), Hippocampus cDNA homologue to Microsomal Signal Peptidase, Neuronal Leucine Rich Repeat Protein 1 (NLRR-1), Synaptotagmin V, Cadherin 1, ESTs weakly similar to KIAA0982 protein or RIKEN cDNA 2310042NO2.

11. The method of claim 1, wherein expression of at least one gene of the two or more genes is diminished in the presence of Nerve Growth Factor.

12. The method of claim 11, wherein the gene is small proline-rich repeat protein 1A, Motopsin (Neurotrypsin), Inhibin Beta B, G protein-coupled receptor 19, Lipocalin 2, or Troponin C.

13. The method of claim 1 wherein the two or more genes comprise Substance P and galanin or Substance P and small proline rich repeat protein 1A (sprr1A).

14. The method of claim 1, wherein the candidate agent is an antibody.

15. The method of claim 1, further comprising the step of isolating RNA from the neurons.

16. The method of claim 15, wherein the isolation utilizes silica based magnetic beads that bind RNA under high salt conditions and wherein the silica based magnetic beads are washed with about 80% ethanol.

17. The method of claim 16, wherein the RNA isolated by said method is detected by polymerase chain reaction using intron spanning primers.

18. The method of claim 1, wherein the candidate agent is dissolved in an organic solvent.

19. The method of claim 18, wherein the organic solvent is dimethyl sulphoxide.

20. A method of assessing the therapeutic potential of a combination therapy comprising Nerve Growth Factor, said method comprising:
(a) contacting a culture of neurons expressing the high-affinity trk A receptor with two or more candidate agents and Nerve Growth Factor, and
(b) measuring the level of expression of at least two NGF responsive genes in said culture, wherein an alteration of the level of expression of the genes indicates the therapeutic potential of the combination therapy.

21. The method of claim 20, wherein the culture of neurons expressing the high-affinity trk A receptor is a primary nociceptive neuronal culture.

22. The method of claim 21, wherein the neurons are dorsal root ganglion neurons or trigeminal ganglion neurons.

23. The method of claim 20, wherein the neurons in the culture are between about 100 to about 200 cells per well or 3.5 cells per square millimeter to about 7 cells per square millimeter.

24. The method of claim 20, wherein the NGF concentration is between about 0.1 ng/ml to about 50 ng/ml.

25. The method of claim 20, wherein the two or more genes comprise Substance P and galanin or Substance P and small proline rich repeat protein 1A (sprr1A).

26. The method of claim 20, wherein at least one of the two or more candidate agents is an antibody.

27. A method of assessing the ability of a candidate agent to modulate NGF activity comprising:
(a) contacting a culture of neurons expressing the high-affinity trk A receptor with a candidate agent and NGF, and
(b) measuring the level of expression of one or more NGF responsive genes in said culture, wherein an alteration of the level of expression of the one or more genes indicates the therapeutic potential of the candidate agent.

28. The method of claim 27, wherein the culture of neurons expressing the high-affinity trk A receptor is a primary nociceptive neuronal culture.

29. The method of claim 28, wherein the neurons are dorsal root ganglion neurons or trigeminal ganglion neurons.

30. The method of claim 27, wherein the neurons in the culture are between about 100- to about 200 cells per well or 3.5 cells per square millimeter to about 7 cells per square millimeter.

31. The method of claim 27, wherein the NGF concentration is between about 0.1 ng/ml to about 50 ng/ml.

32. The method of claim 27, wherein the one or more genes comprise substance P, galanin or sprr 1.
